(19) United States
(12) Patent Application Publication
(10) Pub. No.: US 2009/0227011 A1
(43) Pub. Date: Sep. 10, 2009

(54) METHODS OF PURIFYING PLASMID DNA

(75) Inventor: Shizhong Chen, San Diego, CA (US)

Correspondence Address: KNOBBE MARTENS OLSON & BEAR LLP, 2040 MAIN STREET, FOURTEENTH FLOOR, IRVINE, CA, 92614, US

(73) Assignee: Shizhong Chen, San Diego, CA (US)

(21) Appl. No.: 12/394,397

(22) Filed: Feb. 27, 2009

Related U.S. Application Data
(60) Provisional application No. 61/032,860, filed on Feb. 29, 2008.

(51) Int. Cl. C12N 15/00

(52) U.S. Cl. 435/320.1

(57) ABSTRACT

Described herein are novel methods to purify plasmid DNA from host cells. After a standard lysis procedure that releases plasmid DNA from the host cells, two sequential precipitation procedures separate plasmid DNA from essentially all impurities of the host cells. An optional third precipitation procedure further polishes the plasmid DNA for its most demanding applications, such as for gene therapy or pharmaceutical uses.

### CROSS-REFERENCE TO RELATED APPLICATIONS

This application claims priority to U.S. Provisional Patent Application No. 61/032,860, filed on Feb. 29, 2008, entitled “Methods of Purifying Plasmid DNA.”

### BACKGROUND OF THE INVENTION

1. Field of the Invention

The invention relates to methods for producing plasmid DNA for laboratory applications, as well as clinical applications. The methods are particularly concerned with the use of selective precipitation to separate plasmid DNA from the impurities of the host cells, such as host cell RNA, DNA, and protein.

2. Description of the Related Art

Plasmids are extrachromosomal molecules of DNA found in a variety of host cells and behave as accessory genetic units that replicate independent of the host chromosomes. For its ease of manipulation, propagation, and purification, plasmid DNA has been used for decades in biological research, particularly in molecular biology, as a vector for cloning and transferring genes of interest. In recent years, plasmid DNA has also found increasing use in gene therapy.

Plasmids depend on host enzymes and proteins for their replication. The production of plasmid DNA, therefore, involves the growth of host cells that carry the plasmid, the harvest of the host cells, the lysis of the host cells to release the plasmid, and the purification of plasmid DNA away from other components from the host cells. Traditionally, Escherichia coli (E. coli) bacteria have been a favorite host for growing and purifying plasmid DNA.

For its ease of use and scalability, one method of host cell lysis is alkaline lysis, first developed by Birnboim and Doly. See C. Birnboim and J. Doly, “A Rapid Alkaline Extraction Procedure for Screening Recombinant Plasmid DNA,” Nucleic Acids Research, 7:1513-1523, 1979, the contents of which are hereby incorporated by reference in their entirety. In this method, host cells harvested by centrifugation or filtration are first re-suspended in a buffer solution, traditionally called Solution I (“Sol. I”), and then lysed by adding an alkaline solution, traditionally called Solution II (“Sol. II”). One of the original Sol. I compositions was a mixture of 2 mg/ml lysozyme, 50 mM glucose, 10 mM cyclohexane diamine tetraacetate (CDTA), and 25 mM Tris-HCl, with a solution pH of 8.0, although many other re-suspension solutions are now well known and more preferred. One of the original Sol. II compositions was a mixture of 0.2 N NaOH and 1% sodium dodecyl sulfate (SDS), although any solution capable of lysing host cells can be used.

After Sol. I and Sol. II are added to the host cells, the final pH of the lysate is 12-12.5, which causes denaturation of the bacterial genomic DNA. The covalently closed circular plasmid DNA (ccc-DNA), however, does not denature at this pH. The lysis mixture is then neutralized by adding an acidic neutralizing solution, traditional called Solution III (“Sol. III”) containing a high concentration of certain salts, such as 3 M sodium acetate adjusted to a pH of 4.8 with water and glacial acetic acid. The high salt and SDS form complex with the host protein, and the neutralization causes the host genomic DNA to aggregate and form a precipitation with the protein. While these Sol. III precipitate out and remove majority of the host chromosomal DNA and protein, they do not remove host RNA effectively. A centrifugation or filtration step is then used to remove the genomic DNA-protein aggregate, leaving the plasmid DNA in the clarified lysate. The plasmid DNA can then be precipitated from the clarified lysate by alcohol or polyethylene glycol. See J. T. L is and R. Schleif, “Size Fractionation of Double-Stranded DNA by Precipitation with Polyethylene Glycol,” Nucleic Acids Research, 2:383-389, 1975, the contents of which are hereby incorporated by reference in their entirety.

By testing various salt solutions for Sol. III, precipitation and removal of much of the host genomic DNA and protein has been achievable. See I. Feliciello and G. Chinali, “A Modified Alkaline Lysis Method for the Preparation of Highly Purified Plasmid DNA from Escherichia coli,” Analytical Biochemistry, 212: 394-401, 1993, the contents of which are hereby incorporated by reference in their entirety. However, a clean separation of plasmid DNA from host RNA has been difficult, owning to the abundance of host RNA (in the case of E. coli, the weight to weight ratio of RNA to plasmid DNA exceeds 30) and its similar physical and chemical properties to plasmid DNA.

For laboratory applications, a combination of RNase treatment, as described in Ahn et al., “Rapid Mini-Scale Plasmid Isolation for DNA Sequencing and Restriction Mapping,” BioTecniques, 29: 466-468, 2000, the contents of which are hereby incorporated by reference in their entirety, to degrade the bulk of host RNA and an affinity chromatography step (for example, commercial plasmid purification kits from Qiagen, Invitrogen, Promega and many other vendors) to wash the residual RNA degradation product from plasmid DNA are generally used to obtain relatively pure plasmid. Each of the two treatments, however, has a number of disadvantages. The RNase treatment can interfere with subsequent applications of the plasmid DNA (for example, in vitro RNA transcription). The affinity chromatography resins have binding limits, which requires a large column of resins to purify a large amount of plasmid DNA. For example, to purify plasmid DNA from a 2-liter culture of E. coli, the loading of the clarified lysate to a plasmid binding column by Qiagen takes about four to five hours. Therefore, mid- to large-scale plasmid purification in a laboratory is a slow and costly process.

For clinical applications, it is desirable to completely avoid the RNase treatment because the RNase used are usually animal-derived and may contain viruses and other pathogenic agents. The affinity chromatography is also impractical for the large scale purification of pharmaceutical-grade plasmid DNA, and is replaced by other kinds of chromatography, such as ion-exchange chromatograph, size-exclusion chromatography, or reverse-phase chromatography. The host RNA, however, will bind to these chromatography resins, and interfere with the process. Various other methods have been explored to reduce the RNA content from the clarified lysate before its loading to the chromatography column, including LiCl₂ treatment and CaCl₂ treatment after neutralization. See U.S. Pat. No. 6,410,274 to Bhikhabhai and Eon-Duval et al., “Precipitation of RNA Impurities with High Salt in a Plasmid DNA Purification Process: Use of Experimental Design to Determine Reaction Conditions,” Biotechnology and Bioengineering, 83: 545-552, 2003, the contents of both references are hereby incorporated by reference in their entirety. However, the steps under these methods are performed to the clarified lysate after the neutralization step and can only precipitate a significant portion, but not all of the host RNA from the clarified lysate.

To reduce the cost of pharmaceutical-grade plasmid production, several alternative methods have been explored, such as the aqueous two-phase extraction method (Ribeiro et al., “Isolation of Plasmid DNA from Cell Lysates by aqueous two-phase systems,” Biotechnology and Bioengineering, 78: 376-384, 2003), selective precipitation of plasmid DNA by compaction agents such as spermine and spermidine (Murphy et al., “Purification of Plasmid DNA Using Selective Precipitation by Compaction Agents,” Nature Biotechnology, 17: 822-823, 1999), and fractional precipitation of plasmid DNA by the detergent CTAB (Lander et al., “Fractional Precipitation of Plasmid DNA from Lysate by CTAB,” Biotechnology and Bioengineering, 79: 777-784, 2002). The contents of each of these references are hereby incorporated by reference in their entirety. Each of these methods also has disadvantages. Removal of RNA is incomplete in the technology of the aqueous two-phase extraction method. The spermine and spermidine precipitation have potential safety issues because both spermine and spermidine are known for toxicity in animal and in cell culture. And finally, the CTAB (centyltrimethylammonium bromide) fractional precipitation method only works well within a very narrow range of CTAB and NaCl concentration and may be difficult to perform and scale up.

PEG has been used previously for differential precipitation in plasmid DNA purification. See Paithankar et al., “Precipitation of DNA by Polyethylene Glycol and Ethanol: Nucleic Acid Research, 19: 1345, 1991; U.S. Pat. No. 5,561,064, 1996 to Marquet et al.; Hartley et al., “PEG Precipitation for Selective Removal of Small DNA Fragment,” Focus, 18: 27, 1996; and Schmitz et al., “Purification of Nucleic Acids by Selective Precipitation with Polyethylene Glycol 6000,” Analytic Biochemistry, 354: 311-313, 2006, the contents of all of these documents are hereby incorporated by reference. In these studies, PEG concentration below 5% was found to not precipitate the nucleic acid. Furthermore, U.S. Pat. No. 5,561,064 states that 4% PEG will only selectively precipitate host impurities but not plasmid DNA, and that 10% PEG is needed to precipitate plasmid DNA.

There exists a need for a plasmid DNA purification method that provides a high purity of plasmid DNA with a minimal amount of processing steps.

### SUMMARY OF THE INVENTION

Described herein is a method of purifying plasmid DNA. In an embodiment, the methods described herein use selective precipitation to purify plasmid DNA from host cell impurities. In an embodiment, the method of purifying plasmid DNA from host cells for laboratory or clinical use comprises the steps of a) lysing host cells containing plasmid DNA to obtain a lysate; b) precipitating host cell impurities from the lysate of step a) by adding a first solution comprising at least one monovalent cation and at least one divalent cation; c) centrifuging or filtrating the lysate from step b) to remove the precipitation of the host cell impurities forming a clarified lysate; d) precipitating plasmid DNA from the clarified lysate of step c) by adding a second solution comprising a first plasmid DNA precipitating agent; and e) collecting the plasmid DNA precipitation by a separation step. In an embodiment, the first solution further comprises a second divalent cation.

The methods described herein provide significant advantages over current plasmid purification technologies, which predominantly employ various chromatography methods or RNase treatments to a clarified lysate in order to separate the plasmid DNA from impurities. Some common impurities include host cell RNA, DNA, and/or proteins. In an embodiment, the method of purifying plasmid DNA does not comprise a chromatography step to separate and/or purify the plasmid DNA. For example, in an embodiment, the method of purifying plasmid DNA described herein does not incorporate affinity chromatography, ion-exchange chromatograph, size-exclusion chromatography, and/or reverse-phase chromatography. Use of chromatography methods results in longer plasmid DNA purification times due to the time it takes for the solution to move across the column. Additionally, column separation of plasmid DNA results in lower yield of the desired final product. In an embodiment, the method of purifying plasmid DNA does not comprise an RNase treatment. RNase treatment is particularly undesirable in clinical and pharmaceutical applications, due to the toxicity of the RNase residue. RNase is generally purified from animal sources, such as pancreases, and it is difficult to exclude viral and/or other pathogen contamination. However, any of these aforementioned chromatography steps and/or RNase treatments optionally can be used in the methods described herein, if so desired by one having ordinary skill in the art.

Advantageously, the methods described herein can be used for both the laboratory production of plasmid DNA and the pharmaceutical production of plasmid DNA. For example, method steps a) through e) as set forth above can be employed in the laboratory production of plasmid DNA. However, additional purification, or polishing steps may be employed for the pharmaceutical production of plasmid DNA or in laboratory applications when DNA of high purity is required. In an embodiment, the method of purifying plasmid DNA further comprises f) dissolving the plasmid DNA precipitation collected in step e) in water; g) re-precipitating the plasmid DNA for a second time by adding a third solution comprising a second plasmid DNA precipitating agent and a salt, and h) re-collecting the plasmid DNA precipitation by a separation step. In an embodiment, the third solution comprises a divalent cation. In an embodiment, the third solution comprises CaCl₂. Optionally, the third solution can comprise at least one monovalent cation and/or at least one divalent cation.

Another further advantage of the purification methods taught herein is that said methods use reagents that are generally regarded as safe (GRAS), and do not require the use of chromatographic resin, resulting in minimizing the cost of purification and maximizing the yield for plasmid DNA production.

Furthermore, the methods described herein are scalable for purifying plasmid DNA from a few dozens microliters to hundreds of liters of cell culture, therefore fitting the needs of both laboratory applications and clinical applications. Advantageously, the methods described herein are adaptable to both the laboratory centrifugation process and the industrial process of filtration for separating pure plasmid DNA from host cell impurities.

In an embodiment, the lysing of the host cells is by alkaline lysis, thus forming an alkaline lysate. In an embodiment, the alkaline lysate is neutralized by an acidic solution comprising an acid, a salt of monovalent cation, and a salt of divalent ion. In an embodiment, the acid is selected from weak acids such acetic acid, citric acid, formic acid, boric acid, hydrofluoric acid, and glycine. In an embodiment, the monovalent cation is selected from the group consisting of ammonium ions, lithium ions, sodium ions, potassium ions, cesium ions and rubidium ions. In an embodiment, the divalent cation is selected from the group consisting of manganese ions, calcium ions, zinc ions, copper ions, cobalt ions and nickel ions. Furthermore, a combination of divalent cations can be used, including, for example, a combination of manganese ions and calcium ions. In an embodiment, the first solution comprises manganese chloride. In an embodiment, the first solution comprises calcium chloride.

In an embodiment, the first solution comprises a neutralization buffer. In an embodiment, the neutralization buffer comprises an acetic solution that comprises an acetic salt. In an embodiment, the first solution comprises manganese chloride, acetic acid, and potassium acetate (KAc). In an embodiment, the first solution further comprises calcium chloride. In an embodiment, the first precipitating agent in the second solution comprises polyethylene glycol. In an embodiment, the second solution is added to provide a final concentration of polyethylene glycol below about 10% of the total solution. In an embodiment, the second solution is added to provide a final concentration of polyethylene glycol below about 4% of the total solution. In an embodiment, the second solution is added to provide a final concentration of polyethylene glycol at about 3.5% of the total solution. In an embodiment, the separation step of step e) comprises centrifugation, filtration, or a combination thereof. In an embodiment, the separation step of step h) comprises centrifugation, filtration, or a combination thereof. In an embodiment, the second precipitating agent in the third solution comprises polyethylene glycol.

### BRIEF DESCRIPTION OF THE DRAWINGS

FIG. 1 illustrates a transferring adapter for transferring material between multi-well plates.

### DETAILED DESCRIPTION OF THE INVENTION

The inventors have surprisingly discovered that the purification of plasmid DNA from host impurities can be vastly improved by first precipitating host cell impurities following lysis by addition of a single first solution and then by selective precipitation of plasmid DNA with a polyethylene glycol solution. In an embodiment, this first solution comprises acetic acid and a combination of at least one monovalent cation and at least one divalent cation. In an embodiment, the at least one monovalent cation is selected from the group consisting of ammonium ions, lithium ions, sodium ions, potassium ions, cesium ions, rubidium ions, and combinations thereof. In an embodiment, the at least one divalent cation is selected from the group consisting of manganese ions, calcium ions, zinc ions, copper ions, cobalt ions, nickel ions, and combinations thereof. In an embodiment, the monovalent ion is ammonium ion and the divalent ion is manganese ion. In an embodiment, the monovalent ion is sodium ion and the divalent ion is manganese ion. In an embodiment, the monovalent ion is potassium ion and the divalent ion is manganese ion. In embodiments where the divalent ion is manganese ion, the first solution can further comprise a second divalent ion such as chloride ion.

The inventors have discovered that combining two or more salts, wherein at least one salt comprises monovalent cations and at least one salt comprises divalent cations, such as potassium acetate and manganese chloride, directly to the host cell lysis can precipitate nearly the entirely of host cell RNA, which is an impurity that has been most difficult to remove from plasmid DNA. The inventors have further discovered that adding two different divalent ions, such as calcium and manganese ions, directly to the host cell lysis improves the purity of the resulting plasmid. This discovery significantly lessens the need for further concentrating or purification steps in the plasmid DNA purification process. Any combination of monovalent and divalent ions, including combinations of ammonium ions, sodium ions, potassium ions, and cesium ions with calcium ions, manganese ions, zinc ions, copper ions, cesium ions, cobalt ions, and nickel ions are useful in the methods described herein. For example, one, two, three, or four or more monovalent ions and one, two, three, or four or more divalent ions may be used in combination with one another.

The plasmid used herein can be of any origin and size and can carry any gene of interest. Microbial organisms, such as E. Coli, are most commonly used for growing plasmid, but the use of host cells is not limited. The method of host cell culture, such as in incubators, shakers, fermentor etc., is also not limited, and well known in the art. The culture host cells can be harvested by centrifugation or filtration. The harvest cells can be stored frozen or processed immediately.

In an embodiment, the step of lysing host cells containing plasmid DNA to obtain a lysate can be a traditional alkaline lysis step, which may be performed using a traditional Sol. I and a traditional Sol. II. In an embodiment, the alkaline lysis of host cells step comprises re-suspending harvested host cells by centrifugation or filtration in a buffer solution of Sol. I (e.g., 50 mM Tris-HCl, 10 mM ethylenediamine tetraacetic acid (EDTA), pH 8.0) and then lysing the cells by adding a Sol. II (e.g., 0.2 N NaOH and 1% SDS). The exact formulations for Sol. I and Sol. II are not limited. Sol. I generally provides host cell suspension in the solution creating an isosmotic environment and Sol. II generally provides a strong base in order to lyse the cells. Those having ordinary skill in the art will understand that any commonly known solutions for Sol. I and Sol. II may be used to carry out the lysing step.

To lyse the host cells, the volume ratio of Sol. I to Sol. II is not fixed, and can range anywhere from about 1:2 to about 2:1. In an embodiment, Sol. I and Sol. II with the above described formulations are used at the ratio of about 1:1. The actual volume used of each Solution depends on the amount of cells to be processed, as illustrated in the Examples below. This skilled in the art, guided by the disclosure herein, can optimize the ratio of Sol. I to Sol. II to provide optimal lysing of the host cells.

Following the lysis step, host cell impurities are precipitated from the lysate by adding a first solution comprising salts of at least one monovalent cation and at least one divalent cation. In an embodiment, the first solution comprises potassium and manganese ions. In an embodiment, the first solution comprises potassium ions, manganese ions, and calcium ions. In an embodiment, the addition of the first solution occurs simultaneously with addition of a neutralization buffer. For example, the first solution further comprises a neutralization buffer, such as acetic acid. In an embodiment, the at least one monovalent cation and at least one divalent cation are added directly to Sol. III as two or more salts (e.g., potassium acetate with manganese chloride). In an embodiment, the salts comprise one of ammonium ions, lithium ions, sodium ions, potassium ions, cesium ions and rubidium ions, and one of manganese ions, calcium ions, copper ions, zinc ions, cobalt ions, or nickel ions, although any combination of monovalent and divalent cation can be used in the methods described herein.

Selection of the appropriate anions of the salts can be made by those of ordinary skill in the art. It is desirable that the salts in the first solution be aqueous soluble, so selection of the anion for the salts can be based on the water solubility of the resulting salt. In an embodiment, the salts comprise one of acetate ions, citrate ions, glycine ions, phosphate ions, chloride ions, fluoride ions, iodide ions, or sulfate ions. In an embodiment, the salts are selected from the group consisting of ammonium acetate, lithium acetate, sodium acetate, potassium acetate, cesium acetate, rubidium acetate, lithium citrate, sodium citrate, potassium citrate, cesium citrate, lithium borate, sodium borate, potassium borate, lithium glycine, sodium glycine and potassium glycine, ZnCl₂, CuSO₄, NiSO₄, CaCl₂, MnCl₂. In an embodiment, the two salts are KAc and MnCl₂. In an embodiment, the first solution comprises acetic acid, potassium acetate, and manganese chloride.

In an embodiment, Sol. I and Sol. II can be combined into a single formulation, herein referred to as Solution A (“Sol. A”). A vial of Sol. A can be paired with a vial of Solution B (“Sol. B”) to form a kit for purifying plasmid DNA. In an embodiment, the Sol. B comprises the first solution described herein comprising at least one monovalent cation and at least one divalent cation described herein. Thus, the number of vials needed for plasmid DNA purification can be significantly reduced.

An embodiment provides a kit for the purification of plasmid DNA comprising a Solution I that comprises Tris-HCl and EDTA; a Solution II that comprises NaOH and SDS; and a Solution III that comprises acetic acid, potassium acetate, and manganese chloride. Optionally, Solution III may further comprise calcium chloride. In an embodiment, Solution I comprises 50 mM Tris-HCl and 10 mM EDTA at pH of 8.0; Solution II comprises 0.2 N NaOH and 1% SDS; and Solution III comprises 2.0 M acetic acid, 0.6 M potassium acetate, 0.4 M calcium chloride, and 0.4 M manganese chloride.

Another embodiment provides a kit for the purification of plasmid DNA comprising a Solution A that comprises Tris-HCl, EDTA, NaOH, and SDS and a Solution B that comprises acetic acid, potassium acetate, and manganese chloride. Optionally, Solution B may further comprise calcium chloride. Thus, Solution A can be formed by combining Sol. I and Sol. II as described above. In an embodiment, Solution A comprises 50 mM Tris-HCl, 10 mM EDTA, 0.2 N NaOH and 1% SDS and Solution B comprises 2.0 M acetic acid, 0.6 M potassium acetate, 0.4 M calcium chloride, and 0.4 M manganese chloride.

In an embodiment, the first solution is usable in a kit for plasmid DNA purification. For example, where traditional Sol. I and Sol. II are used to lysate the host cells in a kit, the first solution can comprise Sol. III to create a three-solution kit for plasmid DNA purification. Furthermore, where Sol. A, defined above, is used to lysate the host cells in a kit, the first solution can comprise Sol. B to create a two-solution kit for plasmid DNA purification.

CaCl₂ has been previously used for plasmid DNA purification to reduce host RNA content in the clarified lysate. See Raymond et al., “Large-Scale Isolation of Covalently Closed Circular DNA Using Gel Filtration Chromatography,” Analytical Biochemistry, 173: 125-133, 1988, the contents of which are hereby incorporated by reference in their entirety, and U.S. Pat. No. 6,410,274 to Bhikhabhai. However, the CaCl₂ in these methods was added to the clarified lysate as a separate step to precipitate and reduce the host RNA content. Furthermore, the reduction of impurities was not complete following precipitation with CaCl₂.

In an embodiment, the method according to this invention uses a combination of salts of monovalent and divalent cations, e.g. a combination of potassium and manganese ions, directly in the neutralization buffer immediately following lysis of the host cells. The neutralization buffer disclosed in this invention precipitates the host components much more effectively. A combination of divalent ions, e.g. a combination of calcium and manganese ions, further improves the purity of plasmid. In an embodiment, the first solution is the neutralization buffer. In an embodiment, the neutralization buffer comprises KAc and MnCl₂. In an embodiment, the neutralization buffer comprises KAc, CaCl₂ and MnCl₂.

In an embodiment, a first solution comprising a neutralization buffer is added to cause precipitation of host components. In an embodiment, the first solution comprises acetic acid and an acetic salt. Various acetic salts may be used, such as ammonium acetate, sodium acetate and potassium acetate. The concentration of the acetic acid and acetate salt, such as potassium acetate, in the neutralization buffer can vary over a wide range. In an embodiment, the concentration of acetic acid is about 0.1 M to about 6 M. In an embodiment, the concentration of acetic acid is about 0.5 M to about 5 M. In an embodiment, the concentration of acetic acid is about 1 M to about 4 M. In an embodiment, the concentration of acetic acid is about 2 M to about 3 M.

Acetic salt can be used to provide the at least one monovalent cation. In an embodiment, the concentration of acetate salt is about 0.05 M to about 6 M. In an embodiment, the concentration of acetate salt is about 0.1 M to about 5 M. In an embodiment, the concentration of acetate salt is about 0.4 M to about 3 M. In an embodiment, the concentration of acetate salt is about 0.6 M to about 1 M.

The divalent ions can be provided to the first solution by adding a variety of divalent cation salts. The concentration of the divalent cation salts in the first solution can also vary. Where more than one divalent salt is present in the first solution, the concentration of each can be independently selected. Each divalent ion salt, including ZnCl₂, CuSO₄, NiSO₄, CaCl₂, or MnCl₂ can be present in a concentration of about 0.05 M to about 4 M. In an embodiment, each divalent ion salt is present in a concentration of about 0.1 M to about 2.0 M. In an embodiment, each divalent ion salt is present in a concentration of about 0.2 M to about 1.0 M. In an embodiment, each divalent ion salt is present in a concentration of about 0.4 M to about 0.6 M.

In an embodiment, manganese chloride is present in an amount of about 0.1 M to about 2.0 M. In an embodiment, manganese chloride is present in an amount of about 0.2 M to about 0.8 M. In an embodiment, manganese chloride is present in an amount of about 0.4 M. Other divalent cations may further be included, including calcium cations derived from the salt calcium chloride. In an embodiment, calcium chloride is present in an amount of about 0.1 M to about 2.0 M. In an embodiment, calcium chloride is present in an amount of about 0.2 M to about 1.0 M. In an embodiment, calcium chloride is present in an amount of 0.2 M to about 0.8 M. In an embodiment, calcium chloride is present in an amount of 0.4 M.

In some embodiments, more than one divalent ion may be present. In such embodiments, the ratio, which can be expressed by the relative molarities of the relative salts, of the two divalent ion salts can vary over a wide range. In an embodiment, the ratio of a first divalent salt to a second divalent salt is from about 1:4 to about 4:1. In an embodiment, the ratio of a first divalent salt to a second divalent salt is from about 1:2 to about 2:1. In an embodiment, the ratio of a first divalent salt to a second divalent salt is about 1:1. In an embodiment, the first divalent salt is MnCl₂. In an embodiment, the second divalent salt is CaCl₂.

The volume of the first solution can also vary, and will depend on the actual formula of the first solution. The titration of the first solution is empirical and well within the skill of workers in this art. In an embodiment, the first solution comprises 2.0 M acetic acid, 0.6 M potassium acetate, 0.4 M manganese chloride, and 0.4 M calcium chloride. In an embodiment, the volume of the first solution (i.e. Sol. III) is the same as Sol. I described above.

In an embodiment, after addition of the first solution, a clarified lysate is formed by using a separation step on the lysate to remove the precipitation of the host cell impurities. Various forms of separation can be used. In an embodiment, the separation step comprises filtering. In an embodiment, the separation step comprises centrifuging. In an embodiment, the separation step comprises filtering and centrifuging.

For various laboratory purifications, the precipitation of host components formed after adding the first solution can be spun out by centrifuge. The precipitation can also be filtered out. The high salt density of the alkaline lysate combined with the first solution causes the precipitated host cell impurities to float to the top very quickly, permitting an easy filtration. This filtration method is more suitable for industrial purification of plasmid DNA. The transferring adaptor, described in further below, permits an easy and efficient filtration of the neutralized lysate.

The addition of a second solution comprising a first plasmid DNA precipitating agent then selectively precipitates the plasmid DNA from the clarified lysate. Various types of precipitation agents can be used. Some commonly known precipitating agents include polyethylene glycol and alcohol, such as ethanol and isopropanol. For laboratory purifications, the plasmid DNA precipitation can be centrifuged down. Alternatively, the plasmid DNA precipitation can also be collected via filtration. The plasmid DNA precipitation can be further polished with an additional round of PEG precipitation, as described further below.

In an embodiment, the plasmid DNA is selectively precipitated out of the clarified lysate by adding polyethylene glycol (PEG). The inventors have discovered that the Sol. III disclosed in this invention results in a clarified lysate that will allow the use of surprisingly low concentrations of PEG to selectively precipitate plasmid DNA. The concentration of PEG that can be used to precipitate the plasmid DNA is much lower than previously achieved. In an embodiment, the second solution comprising polyethylene glycol is added in step d) to provide a final concentration of polyethylene glycol below about 10% based on the total weight of the solution. In an embodiment, the second solution comprising polyethylene glycol is added in step d) to provide a final concentration of polyethylene glycol below about 4% based on the total weight of the solution. In an embodiment, the second solution comprising polyethylene glycol is added in step d) to provide a final concentration of polyethylene glycol at about 3.5% based on the total weight of the solution.

When the neutralization buffer described herein comprising at least one monovalent and one divalent ions is employed, PEG final concentration at 3.5% and above is enough to precipitate plasmid DNA, which is a lower concentration of PEG required compared to currently used methods. The higher concentration of PEG necessary in other methods is not desired, however, for it may precipitate unwanted impurities and salt.

The molecular weight of PEG used in this invention is not limited, and can vary from PEG 400 to PEG 8000 and above. For instance PEG 800, PEG 1000, PEG 2000, PEG 4000, PEG 6000, PEG 8000, and PEG 10000 can all be used. In an embodiment, PEG 8000 at final concentration of 4% is used to precipitating the plasmid DNA.

Preferably, the methods described herein are practiced using GRAS reagents. For example, alcohol, which is a commonly known reagent used to precipitate plasmid DNA from clarified lysate, can be replaced by the use of PEG in the methods described herein. However, it is contemplated that alcohol could optionally be used to precipitate the plasmid DNA from the clarified lysate solution. Additionally, the step of rinsing precipitated plasmid DNA pellets can be performed with 70% ethanol. Alternatively, ethanol can be replaced by the use of isopropanol, which further reduces the use combustible reagents in the purification methods described herein. In an embodiment, the precipitating agent comprises isopropanol from about 15% isopropanol to about 50% isopropanol. In an embodiment, the precipitating agent comprises isopropanol from about 20% isopropanol to about 40% isopropanol. In an embodiment, the isopropanol is 30% isopropanol. In an embodiment, the isopropanol is 15% isopropanol.

Upon precipitation of the plasmid DNA from the clarified lysate, a marked improvement in cleanliness of the purified plasmid DNA is observed compared to previous methods. With alkaline lysis methods that do not employ a combination of ions of different valency in the neutralization buffer, the plasmid DNA is precipitated forming white pellets that are usually clearly visible. When the plasmid DNA is precipitated from the clarified lysate according to the methods described herein, no pellets are usually observed or pellets that are observed are translucent.

Occasionally, when a large volume of cells are processed in the purification methods described herein, the shear quantity of plasmid precipitation formed in the 4% PEG 8000 can capture or trap unwanted impurities. In some embodiments, an ultra-high purity may be desirable, such as in clinical or pharmaceutical applications. To ensure the highest purity, the plasmid DNA precipitation can be further polished by dissolving it in water and precipitating a second time with a third solution comprising a salt and a second precipitating agent.

In an embodiment, the salt in the third solution comprises one of ammonium chloride, ammonium acetate, ammonium sulfate, sodium chloride, sodium acetate, sodium sulfate, potassium chloride, potassium acetate, potassium sulfate, magnesium chloride, magnesium acetate, magnesium sulfate, cesium chloride, cesium acetate, or cesium sulfate. In an embodiment, the salt in the third solution comprises calcium chloride. In an embodiment, the second precipitating agent may be the same or different from the first precipitating agent. In an embodiment, the second precipitating agent is PEG 8000.

After the plasmid DNA is dissolved in water, the third solution is added. In an embodiment, the third solution comprises calcium chloride and PEG 8000. The final concentration of the third solution that is added to the dissolved plasmid can be adjusted by those having ordinary skill in the art, guided by the embodiments and examples disclosed herein. In an embodiment, the third solution is added to plasmid dissolved in water at a final concentration of about 40 mM calcium chloride and about 4% PEG.

In an exemplary embodiment, a sequential precipitation of host cell lysate is performed, first by addition with a HAc—KAc—MnCl₂—CaCl₂ buffer, next with addition of a polyethylene glycol (PEG) solution, and then an optional third step of CaCl₂-PEG precipitation, that separates the plasmid DNA from essentially all host components, thus resulting in ultra pure plasmid DNA.

The methods described herein are suitable for plasmid DNA purification from cell culture volume ranging from a few microliters to hundreds of liters. The methods described herein are particularly suitable for high throughput plasmid DNA purification. High throughput plasmid DNA purification can be carried out using multi-well containers, such as 96-well or 384-well microplates. The transferring from one container to another can be done by aspiration using a multi-channel pipette or by a robot.

The process of plasmid DNA purification using the methods described herein can be illustrated by the following steps:
1. Re-suspension of the host cells in Sol. I of 50 mM Tris-HCl, 10 mM EDTA, pH 8.0;
2. Lysis of the cells by adding equal volume of Sol. II of 0.2 M NaOH, 1% SDS;
3. Neutralizing the lysate with equal volume of Sol. III of 2.0 M acetic acid, 0.6 M potassium acetate, 0.4 M manganese chloride, 0.4 M calcium chloride;
4. Obtaining clarified lysate by centrifugation or filtration;
5. Precipitate plasmid DNA from the clarified lysate by adding PEG 8000 to a final concentration of 4%;
6. Collect the plasmid DNA precipitation by centrifugation or filtration;
7. (optional) Dissolve the plasmid DNA in water, and precipitate the plasmid DNA again by adding calcium chloride to a final and concentration of 40 mM and PEG 8000 to a final concentration of 4%. Collect the final plasmid DNA precipitation by centrifugation or filtration.

### Transferring Adaptor

Various problems occur with methods of collecting plasmid DNA. One particular problem arises in the transfer of clarified lysate after centrifugation because the risk of aspirating the pellets of host impurities off from the bottom. It is therefore common practice to not aspirate all of the supernatant, but this leads to a compromised yield of desired product. Where filtration is used to obtain the clarified lysate, the transfer of the neutralized lysate is also a problem because the precipitation formed by host impurities can block the transferring pipette. It is also common practice to leave behind a portion of the neutralized lysate, again leading to a compromised yield of the desired product.

The problems of compromised yield can be overcome by using the methods described herein and a transferring adapter described herein. In an embodiment, a transferring adaptor is crafted to facilitate the simultaneous transfer and filtration of clarified lysate from one originating multi-well plate to another receiving multi-well plate. Multi-well plates are often used in the synthesis and purification of plasmid DNA.

The transferring adapter allows for simultaneous transfer and filtration of clarified lysate from an originating multi-well plate to a receiving multi-well plate. The transferring adaptor described herein preferably has the same multi-channel format on one side to match the originating multi-well plate, and the same multi-channel format on the other side match the receiving multi-well plate. Each transferring channel on the transferring adaptor comprises a top half protrusion and a bottom half protrusion. Preferably, the protrusions have a similar construction, e.g. circular or polygonal, to the corresponding wells on the multi-well plates. However, the shape of the transferring channel is not limited, and it could even be square or triangular. The top half and bottom half of the transfer channels can, but need not be identical. For example, the top protrusion of the transfer channel can be circular, while the bottom protrusion of the transfer channel can be square. Furthermore, the sizes of the top half and bottom half of the transfer channel can be the same or different.

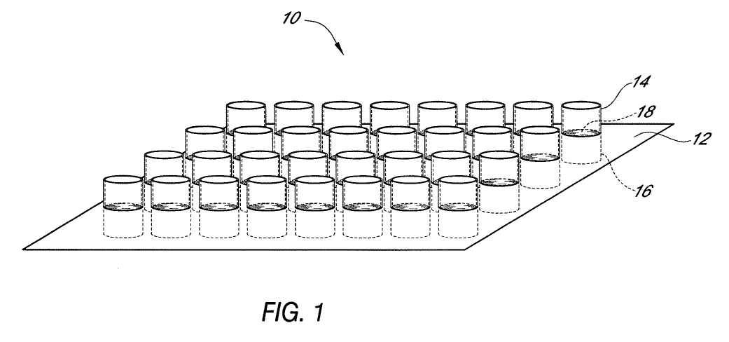

FIG. 1 illustrates a transferring adapter 10 for transferring material between multi-well plates. A flat plate 12 manufactured of plastic or other solid material holds the transferring channel in place. On the side of the transferring adaptor 10 that matches the originating multi-well plate, a plurality of protrusions 14 extend out and correspond to the number of wells on the originating multi-well plate. On the opposite side of the transferring adaptor 10 that matches the receiving multi-well plate, a plurality of protrusions 16 extend out and correspond to the number of wells on the receiving multi-well plate. One top half protrusion 14 and one bottom half protrusion 16 together form a single transferring channel.

The protrusions 14 are constructed to form water-tight seals with at least the originating multi-well plate, and optionally the receiving multi-well plate, when the adaptor is placed over a multi-well plate. These water-tight seals may be formed by providing the ends of the protrusions with tapered outer edges. The tapered outer edges should allow the protrusions of the transferring adapter to fit snugly into the wells of the multi-well plates. These water-tight seals may also be formed by providing the protrusions with tapered inner edges. The tapered inner edges should allow protrusions from the wells of the originating multi-well plate to fit inside. Optionally, the protrusions 16 on the side of the transferring adapter that correspond with the receiving multi-well plate can also have tapered edges to provide a water-tight fit with the wells.

Although the transferring adapter 10 in FIG. 1 shows a 4×8 cylinder configuration, other configurations are contemplated. For example, the array on the transferring adapter can be 8×12, 16×24, 32×48, or any other number that allows the device to transfer material from one multi-plate well to another multi-plate well.

In the center of a transferring channel is a filter membrane 18. In an embodiment, each transferring channel comprises a filter membrane. The filter membrane can comprise any number of structures suitable for filtration. In an embodiment, the filter membrane is constructed with a porous plastic, a porous ceramic material, a steel screen, filter paper, cellulose cloth, or a combination thereof. Any commonly used synthetic or natural materials can comprise the filter material, and those having ordinary skill in the art, guided by the disclosure herein, can select an appropriate filter membrane. The filter membrane can be hydrophilic or hydrophobic. The filter membrane may be enforced from the receiving side by plastic or other solid support. The pore size of the membrane can vary depending on the use of the adaptor.

For transferring clarified lysate after centrifugation, the pore size of the membrane can be large, e.g., up to 1 millimeter. In an embodiment, the pore size of the membrane is about 0.1 microns to about 1 millimeter. In an embodiment, the pore size of the membrane is about 1 micron to about 500 microns. In an embodiment, the pore size of the membrane is about 5 microns to about 100 microns. In an embodiment, the pore size of the membrane is about 10 microns to about 50 microns.

The transferring adaptor is first fitted onto an originating multi-well plate containing clarified lysate, and then a receiving multi-well plate is fitted, upside down, to the other side of the transferring adaptor. Preferably, the number of protrusions extending from a side of the transferring adaptor matches the number of wells in the multi-well plate. For example, where a 96-well plate is the originating multi-well plate, the number of protrusions on the transferring adaptor will also be 96 on one side, and preferably 96 on the other side to match the number of wells on the receiving multi-well plate.

The whole assembly of originating multi-well plate, transferring adaptor, and receiving multi-well plate is then flipped over, causing the clarified lysate to flow from the originating multi-well plate to the receiving multi-well plate, and the membrane filter mesh catches and stops pellets from flowing into the new plate.

For obtaining clarified lysate via filtration, the transferring adaptor can be used to combine centrifugation with filtration. In this circumstance, the pore size of the filter membrane should be smaller, e.g., not larger than about 20 microns, preferably not larger than 10 microns, and preferably is about 5 microns. This said adaptor is first fitted onto the multi-well plate containing the neutralized lysate, and then a new multi-well plate is fitted, upside down, to the other side of the adaptor. The whole assembly comprising the original multi-well plate, transferring adaptor, and new multi-well plate is then flipped over, allowed to stand for several minutes, and then centrifuged. The centrifugal force will cause the lysate to be filtered through the membrane in the transferring adaptor.

The transferring adaptor can be constructed from any material, but preferably with disposable material such as polypropylene, polystyrene or other synthetic materials. The transferring adaptor can also be used for other liquid transfer or filtration application, such as in recombinant protein purification. The mechanisms of transfer can vary and include any known filtering technique. For example, the liquid can flow down by gravity, or the transfer can be facilitated by centrifugation, facilitated by pressure, or facilitated by vacuum.

An embodiment provides a transferring adaptor for simultaneous transfer and filtration of clarified lysate comprising a plurality of top-half protrusions extending out and corresponding to a number of wells on an originating multi-well plate; a plurality of bottom-half protrusions extending out and corresponding to a number of wells on a receiving multi-well plate; wherein the plurality of top-half protrusions have tapered ends that allow the protrusions to form a water-tight seal with the wells of the originating multi-well plate; wherein at least one top half protrusion and at least one bottom half protrusion are together to form a single transferring channel that comprises a filter membrane.

In an embodiment, the host cells described in the methods provided herein are present in a multi-well container and/or are processed in a multi-well container. In an embodiment, a transferring adaptor is used to transfer and filter the host cells of the multi-well lysates from an originating multi-well container to a receiving multi-well container. In an embodiment, the transferring adaptor described herein is used in the methods of purifying plasmid DNA described herein.

### EXAMPLES

Particular aspects of the invention may be more readily understood by reference to the following examples, which are intended to exemplify the invention, without limiting its scope to the particular exemplified embodiments. Each of the steps described in the Examples below can be incorporated, without limitation, into the methods described herein. Each of the solutions described in the Examples below, e.g., Sol. I, Sol. II, Sol. A, and Sol. III/Sol. B is intended to be within the scope of the solutions generally described herein.

### Example 1

### Preparation of Plasmid DNA Template for Sequencing from E. coli

Plasmid DNA is the most commonly used DNA template for DNA sequencing, and high throughput (e.g., 96-well to 384-well) purification of plasmid DNA as a sequencing template represents a special case for plasmid DNA purification. First, the amount of templates needed is much less than other plasmid-related applications. Plasmid DNA obtained from even a single E. coli colony on a culture plate is enough for sequencing. It is therefore possible to purify enough plasmid DNA as sequencing template from just a few dozen microliters of cell culture. Second, DNA sequencing from plasmid template is somewhat tolerant to host genomic DNA and protein but sensitive to interference from host RNA. The removal of RNA is therefore important.

Using the methods described herein, plasmid DNA can be easily purified from 20-200 microliters of cell culture. An example of purifying plasmid DNA from E. coli for DNA sequencing is provided below:
1. Inoculate E. coli colonies containing desired plasmids into 96-well culture blocks containing 500 microliters of appropriate culture media. Grow overnight at 37° C. with vigorous shaking;
2. Using a multiple channel pipettor or a robot, transfer up to 250 microliters of overnight culture into a 96-well PCR plate with chimney or raised rim;
3. Pellet the bacteria by centrifugation at 3,000 g for 5 min. Flip the plate to discard the supernatant;
4. Vortex the PCR plate to dislodge and re-suspend the bacterial pellet in the residue culture media.
5. Using a multiple channel pipettor or a robot, dispense 150 microliters of Sol. A [Prepared as a stock solution by mixing equal volumes of (50 mM Tris-HCl, 10 mM EDTA, pH 8.0) and (0.2M NaOH and 1.0% SDS)] to the bacterial resuspension. This should cause the bacteria to lyse and result in a clear solution. If the solution is cloudy, pipet up and down a few times to lyse the bacteria.
6. Using a multiple channel pipettor or a robot, dispense 75 microliters of Sol. III [2.0M HAc, 0.6M KAc, 0.4 M MnCl₂, 0.4 M CaCl₂]. Pipet up and down a few times to mix;
7. Spin down the precipitation by centrifugation at 4,000 g for 15 min;
8. Dispense 25 microliters of 40% PEG 8000 into the wells of a V-bottom 96-well collection plate;
9. Place a transferring adaptor over the PCR plate, and place the V-bottom 96-well collection plate over the transferring adaptor. Flip the whole assembly together to collect the clarified lysate into the V-bottom 96-well collection plate.
10. Alternatively, place 96-well collection plate over the 96-well PCR plate. The chimney or raised rim should fit into the V-bottom 96-well;
11. Flip the two 96-well plate together. The clarified lysate is now transferred into the collection plate. Seal the collection plate with an adhesive seal. Vortex the collection plate to mix the clarified lysate with the 40% PEG 8000;
12. Spin down the plasmid DNA by centrifugation at 4,000 g for 15 min. Remove the seal and flip the plate to discard the supernatant;
13. Add 250 microliters of 15% isopropanol to each well. Let the plate sit for one minute. Flip the plate to discard the isopropanol;
14. Place the plate upside down in a centrifuge, and spin at 200 g for 2 min to remove the residual isopropanol;
15. Add 10-20 microliters of water to dissolve the plasmid DNA.

### Example 2

### Mini-Preparation of Plasmid DNA from 96-Well Culture of E. coli

High throughput purification of plasmid is widely used in biological and pharmacological researches, and is mostly performed by affinity chromatography-based method. The current invention provides a fast and less costly alternative. An example of purifying plasmid DNA according to the methods described herein from 96-well culture of E. coli is provided below:
1. Inoculate E. coli colonies containing appropriate plasmids into 96-well culture blocks containing 1.2 microliters of appropriate culture media. Grow overnight at 37° C. with vigorous shaking;
2. Pellet the bacteria by centrifugation at 3,000 g for 5 min. Discard the supernatant;
3. Vortex the 96-well block to dislodge and re-suspend the bacterial pellets in the residue media.
4. Dispense 240 microliters of Sol. A [Prepared as a stock solution by mixing equal volumes of (50 mM Tris-HCl, 10 mM EDTA, pH 8.0) and (0.2M NaOH and 1.0% SDS)] into the wells.
5. Using a multiple channel pipettor or a robot, dispense 120 microliters of Sol. III [2.0M HAc, 0.6M KAc, 0.4M MnCl₂, 0.4 M CaCl₂]. Cover the plate with an adhesive seal and invert the plate multiple times to mix;
6. Remove the seal and place a transferring adaptor with 5 micron membrane over the 96-well plate. Place a new 96-well plate over the transferring adaptor. Flip the whole assembly over and centrifuge the whole assembly at 1,000 g for 5 minutes. Disregard the top plate as well as the transferring adaptor;
7. Dispense 40 microliters of 40% PEG 8000 into the wells the 96-well collection plate. Vortex to mix the clarified lysate with the PEG;
8. Place a transferring adaptor with 0.1 micron membrane over the 96-well plate. Place a new 96-well plate over the transferring adaptor. Flip the whole assembly over and centrifuge the whole assembly at 3,000 g for 5 minutes. Discard the top (originating) plate, leave the transferring plate on the bottom plate;
9. Add 500 microliters of 15% isopropanol to the wells of the transferring plate, spin at 1,000 g for 2 min.;
10. Replace the bottom plate with a new 96-well plate. Add 50 microliters of water in the wells of the transferring plate. Wait for 1 min, then spin at 1,000 g for 2 min. The dissolved DNA is collected in the bottom plate.

### Example 3

### Mini-Preparation of Plasmid DNA from E. coli

Mini-preparation of plasmid from a small volume of cell culture is a highly-practiced technique in molecular biology. An example of mini-preparation of plasmid DNA according to the methods descried herein from 1.5 ml culture of E. coli is provided below:
1. Inoculate 2 ml culture medium with a single E. coli colony containing appropriate plasmids. Grow overnight at 37° C. with vigorous shaking;
2. Transfer 1.5 ml of the bacteria culture into a microfuge tube. Centrifuge at 8,100 g for 1 min. Discard the supernatant;
3. Add 150 microliters of Sol. I [50 mM Tris-HCl, 10 mM EDTA, pH 8.0] into the tubes. Vortex to re-suspend the pellet;
4. Add 150 microliters of Sol. II [0.2M NaOH and 1% SDS] to the bacterial suspension. Close the tube and mix the contents by inverting the tube several times to lyse the bacteria;
5. Add 150 microliters of Sol. III [2.0 M HAc, 0.6M KAc, 0.4 M MnCl₂, 0.4 M CaCl₂] to the tube. Close the cap and mix the content by inverting the tube several times. A white precipitation should form;
6. Centrifuge the bacterial lysate in a microfuge at full speed for 1-2 min. Transfer the supernatant to a new tube;
7. Add 50 microliters of 40% PEG 8000 to the clarified lysate. Close the tube and invert a few times to mix the contents;
8. Centrifuge the tube in a microfuge at full speed for 1-2 min. Surprisingly, and unlike other plasmid purification protocols, no visible pellet is seen after the centrifuge, showing the cleanness of this protocol. Disregard the supernatant;
9. Add 500 microliters of 15% isopropanol to the tube. Close the tube and invert the tube a few times. Disregard the liquid;
10. Briefly spin the tube in a microfuge to collect all liquid to the bottom. Aspirate all the liquid using a pipette;
11. Add 50-100 microliter of water to dissolve the plasmid DNA.

### Example 4

### Mid-Scale Preparation of Plasmid DNA from E. coli

An example of Mid-scale preparation of plasmid DNA from up to 30 ml culture of E. coli is provided below:
1. Inoculate 5 to 30 ml culture medium with a single E. coli colony containing appropriate plasmids. Grow overnight at 37° C. with vigorous shaking;
2. Spin down the bacteria at 4,500 g for 5 min. Discard the supernatant;
3. Add 600 microliters of Sol. I [50 mM Tris-HCl, 10 mM EDTA, pH 8.0] into the tubes. Vortex to re-suspend the pellet;
4. Transfer the bacteria into a 2 ml microfuge tube. Add 600 microliters of Sol. II [0.2M NaOH and 1% SDS]. Close the tube and mix the contents by inverting the tube until the bacteria are completely lysed;
5. Add 600 microliters of Sol. III [2.0M HAc, 0.6M KAc, 0.4M MnCl₂, 0.4 M CaCl₂] to the tube. Close the cap and mix the content by inverting the tube several times. A white precipitation should form;
6. Centrifuge the bacterial lysate in a microfuge at full speed for 10 min. Transfer the supernatant to a new 2 ml microfuge tube;
7. Add 200 microliters of 40% PEG 8000 to the clarified lysate. Close the tube and invert a few times to mix the contents;
8. Centrifuge the tube in a microfuge at full speed for 5 min. A translucent pellet can be seen, showing the cleanness of this protocol. Disregard the supernatant;
9. In an optional step, add 900 microliters of water to the tube. Vortex to dissolve the plasmid DNA. Add 100 microliters of 0.4 M calcium chloride in 40% PEG 8000 to the tube. Close the tube and invert several times to mix the content. Centrifuge the tube in a microfuge for 2 minutes to pellet the plasmid DNA;
10. Add 500 microliters of 15% isopropanol to the tube. Close the tube and invert the tube a few times. Disregard the liquid;
11. Briefly spin the tube in a microfuge to collect all liquid to the bottom. Aspirate all the liquid using a pipette;
12. Add 100-200 microliter of water to dissolve the plasmid DNA.

### Example 5

### Maximum-Preparation of Plasmid DNA from E. coli

An example of maxipreparation of plasmid DNA from 100 ml culture of E. coli is provided below:
1. Grow 100 ml E. coli culture overnight at 37° C. with vigorous shaking;
2. Transfer the culture to a centrifuge tube and spin down the bacteria at 4,500 g for 5 min. Discard the supernatant;
3. Re-suspend the bacteria in 6 ml of Sol. I [50 mM Tris-HCl, 10 mM EDTA, pH 8.0];
4. Lyse the bacteria by adding 6 ml of Sol. II [0.2M NaOH and 1% SDS];
5. Neutralize the lysate with 6 ml of Sol. III [2.0M HAc, 0.6M KAc, 0.4M MnCl₂, 0.4 M CaCl₂];
6. Centrifuge the lysate at 13,000 g for 10 min (see alternative protocol below at 24). Transfer the supernatant to a new tube;
7. Add 2 ml of 40% PEG 8000 to the clarified lysate. Close the cap and mix the contents. Centrifuge the tube at 13,000 g for 10 min. Disregard the supernatant;
8. Dissolve the pellet in 0.9 ml of water and transfer to a 1.7 ml microfuge tube. Add 100 microliters of 0.4 M calcium chloride in 40% PEG 8000 to the tube. Close the tube and invert several times to mix the content. Centrifuge the tube in a microfuge for 5 minutes to pellet the plasmid DNA;
9. Add 1.5 ml of 15% isopropanol to the tube. Close the tube and invert the tube a few times. Disregard the liquid;
10. Briefly spin the tube in a microfuge to collect all liquid to the bottom. Aspirate all the liquid using a pipette;
11. Add 200-500 microliter of water to dissolve the plasmid DNA;
12. Alternatively, the bacterial lysate can be processed by filtration: transfer the lysate to a syringe that has a 5 micron syringe filter attached. Wait for 2 minutes for the white aggregate flow to the top. Push the lysate through the syringe filter into a new tube;
13. Add 2 ml of 40% PEG 8000 to the clarified lysate. Close the cap and mix the contents;
14. Transfer the lysate to a syringe that has a 0.1 micron syringe filter attached. Push the mixture through the syringe filter to capture the plasmid DNA precipitation on the filter;
15. Remove the syringe plunger. Add 1.0 ml of water to the syringe, and push through with the plunger into a 1.7 ml microfuge tube. Repeat the procedure again, but with 0.45 ml of water, and collect the elution into the same tube;
16. Add 135 microliters of 0.4 M calcium chloride in 40% PEG 8000 to the tube. Close the tube and invert several times to mix the content. Centrifuge the tube in a microfuge for 5 minutes to pellet the plasmid DNA;
17. Add 1.5 ml of 15% isopropanol to the tube. Close the tube and invert the tube a few times. Disregard the liquid;
18. Briefly spin the tube in a microfuge to collect all liquid to the bottom. Aspirate all the liquid using a pipette;
19. Add 200-500 microliter of water to dissolve the plasmid DNA.

### Example 6

### Mega-Preparation of Plasmid DNA from E. coli

An example of mega-preparation of plasmid DNA from 500 ml culture of E. coli is provided below:
1. Grow 500 ml E. coli culture overnight at 37° C. with vigorous shaking;
2. Spin down the bacteria at 4,500 g for 5 min. Discard the supernatant;
3. Re-suspend the bacteria in 24 ml of Sol. I [50 mM Tris-HCl, 10 mM EDTA, pH 8.0];
4. Lyse the bacteria by adding 24 ml of Sol. II [0.2M NaOH and 1% SDS];
5. Neutralize the lysate with 24 ml of Sol. III [2.0M HAc, 0.6M KAc, 0.4M MnCl₂, 0.4 M CaCl₂];
6. Centrifuge the lysate at 13,000 g for 10 min (see alternative protocol below at 24). Transfer the supernatant to a new tube;
7. Add 8 ml of 40% PEG 8000 to the clarified lysate. Close the cap and mix the contents. Centrifuge the tube at 13,000 g for 10 min. Disregard the supernatant;
8. Dissolve the pellet in 1.8 ml of water and transfer to a 2.0 ml microfuge tube. Add 200 microliters of 0.4 M calcium chloride in 40% PEG 8000 to the tube. Close the tube and invert several times to mix the content. Centrifuge the tube in a microfuge for 5 minutes to pellet the plasmid DNA;
9. Add 2.0 ml of 15% isopropanol to the tube. Close the tube and invert the tube a few times. Disregard the liquid;
10. Briefly spin the tube in a microfuge to collect all liquid to the bottom. Aspirate all the liquid using a pipette;
11. Add 0.5 to 1.0 ml microliter of water to dissolve the plasmid DNA;
12. Alternatively, the bacterial lysate can be processed by filtration: transfer the lysate to a syringe that has a 5 micron syringe filter attached. Wait for 2 minutes for the white aggregate flow to the top. Push the lysate through the syringe filter into a new tube;
13. Add 8 ml of 40% PEG 8000 to the clarified lysate. Close the cap and mix the contents;
14. Transfer the lysate to a syringe that has a 0.1 micron syringe filter attached. Push the mixture through the syringe filter to capture the plasmid DNA precipitation on the filter;
15. Remove the syringe plunger. Add 0.9 ml of water to the syringe, and push through with the plunger into a 1.7 ml microfuge tube. Repeat the procedure again and collect the elution into the same tube;
16. Add 200 microliters of 0.4 M calcium chloride in 40% PEG 8000 to the tube. Close the tube and invert several times to mix the content. Centrifuge the tube in a microfuge for 5 minutes to pellet the plasmid DNA;
17. Add 2.0 ml of 15% isopropanol to the tube. Close the tube and invert the tube a few times. Disregard the liquid;
18. Briefly spin the tube in a microfuge to collect all liquid to the bottom. Aspirate all the liquid using a pipette;
19. Add 1 ml of water to dissolve the plasmid DNA.

### Example 7

### Giga-Preparation of Plasmid DNA from E. coli

An example of giga-preparation of plasmid DNA from up to 2.5 liter culture of E. coli is provided below:
1. Grow 2.5 liters of E. coli culture overnight at 37° C. with vigorous shaking;
2. Transfer the culture to a centrifuge tube and spin down the bacteria at 4,500 g for 5 min. Discard the supernatant;
3. Re-suspend the bacteria in 120 ml of Sol. I [50 mM Tris-HCl, 10 mM EDTA, pH 8.0];
4. Lyse the bacteria by adding 120 ml of Sol. II [0.2M NaOH and 1% SDS];
5. Neutralize the lysate with 120 ml of Sol. III [2.0M HAc, 0.6M KAc, 0.4M MnCl₂, 0.4 M CaCl₂];
6. Centrifuge the lysate at 13,000 g for 10 min (see alternative protocol below at 24). Transfer the supernatant to a new tube;
7. Add 40 ml of 40% PEG 8000 to the clarified lysate. Close the cap and mix the contents. Centrifuge the tube at 13,000 g for 10 min. Disregard the supernatant;
8. Dissolve the pellet in 3.6 ml of water and transfer to two 2 ml microfuge tube. Add 200 microliters of 0.4 M calcium chloride in 40% PEG 8000 to each tube. Close the tubes and invert several times to mix the content. Centrifuge the tubes in a microfuge for 5 minutes to pellet the plasmid DNA;
9. Add 2.0 ml of 15% isopropanol to the tubes. Close the tubes and invert the tubes a few times. Disregard the liquid;
10. Briefly spin the tube in a microfuge to collect all liquid to the bottom. Aspirate all the liquid using a pipette;
11. Add 1 ml of water to each tube to dissolve the plasmid DNA and combine the contents of the two tubes;
12. Alternatively, the bacterial lysate can be processed by filtration: transfer the lysate to a syringe that has a 5 micron syringe filter attached. Wait for 2 minutes for the white aggregate flow to the top. Push the lysate through the syringe filter into a new tube;
13. Add 40 ml of 40% PEG 8000 to the clarified lysate. Close the cap and mix the contents;
14. Transfer the lysate to a syringe that has a 0.1 micron syringe filter attached. Push the mixture through the syringe filter to capture the plasmid DNA precipitation on the filter;
15. Remove the syringe plunger. Add 1.8 ml of water to the syringe, and push through with the plunger into a 2.0 ml microfuge tube. Repeat the procedure again and collect the elution into another tube;
16. Add 200 microliters of 0.4 M calcium chloride in 40% PEG 8000 to each tube. Close the tubes and invert several times to mix the content. Centrifuge the tubes in a microfuge for 5 minutes to pellet the plasmid DNA;
17. Add 2 ml of 15% isopropanol to the tube. Close the tube and invert a few times. Disregard the liquid;
18. Briefly spin the tube in a microfuge to collect all liquid to the bottom. Aspirate all the liquid using a pipette;
19. Add 1 ml of water to each tube to dissolve the plasmid DNA. Combine the contents of the two tubes.

### Example 8

### Large Scale Production of Plasmid DNA from E. coli

An example of plasmid DNA from 20 liter culture of E. coli is provided below:
1. Grow 20 liter of E. coli culture in a fermenter;
2. Add 20 grams of Celpure filter aid to the culture and filter the culture through a 10 inch Buchner funnel fitted with a ten inch Whatman™ No. 1 filter;
3. The Celpure-bacteria cake was removed from the Buchner filter and suspended in 1 liter of Sol. I [50 mM Tris-HCl, 10 mM EDTA, pH 8.0] in a large carboy;
4. One liter of Sol. II [0.2M NaOH and 1% SDS] is added to the carboy. The carboy is repeatedly inverted to lyse the bacteria;
5. One liter of Sol. III [2.0M HAc, 0.6M KAc, 0.4M MnCl₂, 0.4 M CaCl₂] is added to the carboy. The carboy is repeatedly inverted;
6. The lysate is filtered through another Buchner funnel fitted with Whatman™ No. 1 filter pre-loaded with 10 grams of Celpure filter aid;
7. Add 340 ml of 40% PEG 8000 to the clarified lysate and mix thoroughly;
8. Filter the mixture through a 142 mm 0.1 micron Millipore™ MCE filter membrane fitted on a 6 inch Buchner funnel. Discard the filtrate;
9. Add 36 ml of water to the filter and collect the filtrate. Add another 36 ml of water to the filter and collect the filtrate.
10. Transfer the filtrate to a centrifuge tube. Add 8 ml of 0.4 calcium chloride in 40% PEG 8000 to the tube. Close the tube and invert several times to mix the content. Centrifuge the tube in a microfuge for 10 minutes to pellet the plasmid DNA;
11. Add 100 ml of 15% isopropanol to the tube. Close the tube and invert the tube a few times. Disregard the liquid;
12. Spin the tube in at 1,000 g for 3 minutes to collect all liquid to the bottom. Aspirate all the liquid using a pipette;
13. Add 20 ml water to dissolve the plasmid DNA.

All references cited herein are incorporated herein by reference in their entirety. To the extent publications and patents or patent applications incorporated by reference contradict the disclosure contained in the specification, the specification is intended to supersede and/or take precedence over any such contradictory material.

The term “comprising” as used herein is synonymous with “including,” “containing,” or “characterized by,” and is inclusive or open-ended and does not exclude additional, unrecited elements or method steps.

All numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the preferred embodiments. At the very least, and not as an attempt to limit the application of the doctrine of equivalents to the scope of the claims, each numerical parameter should be construed in light of the number of significant digits and ordinary rounding approaches.

The above description discloses several methods and materials of the preferred embodiments. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention as embodied in the attached claims.

What is claimed is:

1. A method of purifying plasmid DNA from host cells for laboratory or clinical use, comprising the steps of:
a) lysing host cells containing plasmid DNA to obtain a lysate;
b) precipitating host cell impurities from the lysate of step a) by adding a first solution comprising at least one monovalent cation and at least one divalent cation;
c) centrifuging or filtrating the lysate from step b) to remove the precipitation of the host cell impurities forming a clarified lysate;
d) precipitating plasmid DNA from the clarified lysate of step c) by adding a second solution comprising a first plasmid DNA precipitating agent; and
e) collecting the plasmid DNA precipitation by a separation step.

2. The method according to claim 1, further comprising the steps of:
f) dissolving the plasmid DNA precipitation collected in step e) in water;
g) re-precipitating the plasmid DNA for a second time by adding a third solution comprising a salt and a second plasmid DNA precipitating agent, and
h) re-collecting the plasmid DNA precipitation by a separation step.

3. The method according to claim 1, wherein the lysing of the host cells is by alkaline lysis, thus forming an alkaline lysate.

4. The method according to claim 3, wherein the alkaline lysate is neutralized by an acid solution comprising the at least one monovalent cation and the at least one divalent cation.

5. The method according to claim 4, wherein the acid solution comprises acetic acid, potassium acetate, and manganese chloride.

6. The method according to claim 3, wherein the alkaline lysate is neutralized by an acid solution comprising one monovalent cation and two divalent cations.

7. The method according to claim 6, wherein the acid solution comprises acetic acid, potassium acetate, calcium chloride, and manganese chloride.

8. The method according to claim 1, wherein the at least one divalent cation is selected from the group consisting of calcium ions, manganese ions, zinc ions, copper ions, cesium ions, cobalt ions, and nickel ions.

9. The method according to claim 1, wherein the first solution comprises potassium acetate, calcium chloride, and manganese chloride.

10. The method according to claim 1, wherein the first solution comprises a neutralization buffer.

11. The method according to claim 10, wherein the neutralization buffer comprises acetic acid.

12. The method according to claim 11, wherein the first solution comprises manganese chloride, acetic acid, and potassium acetate.

13. The method according to claim 1, wherein the first precipitating agent in the second solution comprises polyethylene glycol.

14. The method according to claim 13, wherein the second solution is added to provide a final concentration of polyethylene glycol below about 10% of the total solution.

15. The method according to claim 1, wherein the separation step of step e) comprises centrifugation, filtration, or a combination thereof.

16. The method according to claim 2, wherein the separation step of step h) comprises centrifugation, filtration, or a combination thereof.

17. The method according to claim 2, wherein the second precipitating agent in the fourth solution comprises polyethylene glycol.

18. A transferring adaptor for simultaneous transfer and filtration of clarified lysate comprising:
a plurality of top-half protrusions extending out and corresponding to a number of wells on an originating multi-well plate;
a plurality of bottom-half protrusions extending out and corresponding to a number of wells on a receiving multi-well plate;
wherein the plurality of top-half protrusions have tapered ends that allow the protrusions to form a water-tight seal with the wells of the originating multi-well plate; and
wherein at least one top half protrusion and at least one bottom half protrusion are together to form a single transferring channel that comprises a filter membrane.

19. A kit for the purification of plasmid DNA comprising:
a Solution A that comprises Tris-HCl, EDTA, NaOH, and SDS; and
a Solution B that comprises acetic acid, potassium acetate, manganese chloride, and optionally calcium chloride.

20. The kit according to claim 19, wherein
Solution A comprises 50 mM Tris-HCl, 10 mM EDTA, 0.2 N NaOH and 1% SDS; and
Solution B comprises 2.0 M acetic acid, 0.6 M potassium acetate, 0.4 M calcium chloride, and 0.4 M manganese chloride.
